(19) United States
(12) Patent Application Publication
(10) Pub. No.: US 2016/0255884 A1
(43) Pub. Date: Sep. 8, 2016

(54) DEVICE FOR COOLING THE OUTER SURFACE OF A BODY

(71) Applicant: B&C CRYOINNOV, Saint-Gregoire (FR)

(72) Inventors: Guy Chaumont, Leon (FR); Jose Fernandez Sampedro, Montgermont (FR); Erwann Le Bars, La Chapelle des Fougeretz (FR)

(21) Appl. No.: 15/028,679

(22) PCT Filed: Oct. 6, 2014
(86) PCT No.: PCT/FR2014/052528

§ 371 Date: Apr. 11, 2016

(30) Foreign Application Priority Data
Oct. 10, 2013 (FR) 1359870

(51) Int. Cl. A41D 13/005; A01K 13/00; F41H 1/02; A41F 9/00; A41D 31/00; A41D 13/05; A41D 13/06; A41D 1/06; A01K 29/00; A42B 3/28

(52) U.S. Cl. CPC A41D 13/0053; A01K 29/00; A01K 13/008; F41H 1/02; A42B 3/285; A41D 31/0038; A41D 13/0512; A41D 13/065; A41D 1/06; A41F 9/002

(57) ABSTRACT

A device is provided for covering and/or protecting at least one part of the outer surface of a human or animal body. Such a device includes at least one fixed portion for cooling at least one region of the part of the outer surface of the body, able to be refrigerated and/or frozen, and presenting at least three layers arranged in the following order: a breathable layer intended to be applied on and/or near the region; a flexible cold storage and restitution layer; and a heat insulation layer, intended to delay heating of the cold storage and restitution layer.

### 1. CROSS-REFERENCE TO RELATED APPLICATIONS

This Application is a Section 371 National Stage Application of International Application No. PCT/FR2014/052528, filed Oct. 6, 2014, the content of which is incorporated herein by reference in its entirety, and published as WO 2015/052421 on Apr. 16, 2015, not in English.

### 2. FIELD OF THE INVENTION

The field of the invention is that of controlling the body temperature, particularly in view of physiological comfort, of a mammal and/or protecting against heat.

More specifically, the invention relates to body covering and/or protecting devices, such as, in particular, clothing, designed to participate in maintaining the normal skin temperature of a person or an animal, or at least in cooling the body.

It should be noted that in the context of the invention, the term “clothing” is understood to refer to any object intended to cover at least part of the body to conceal it, protect it and/or dress it, such as for example a suit, an accessory, a shoe, a head gear or a covering.

A device according to the invention can, for example, be used in the context of a sports activity or a professional practice, or in a hospital setting, during medical intervention, during the administration of emergency measures or as a precautionary measure.

In certain cases it may be used as fire protection.

### 3. PRIOR ART

Feeling a sensation of discomfort under clothing or equipment is frequent when it is hot, or in the context of physical and/or sports activity sustained.

This discomfort is all the more critical when the clothing or equipment is thick, and/or presents an insulating or cushioning layer, for example to protect against shock, heat, cold or bad weather.

Prolonged exposure to high temperatures or to stress could even be traumatic and/or dangerous for the health, if it causes hyperthermia, also commonly called “heat stroke.”

To respond to this problem, clothing and/or equipment presenting naturally ventilated air passages has been proposed.

One disadvantage of this known technique is that it generally does not enable the skin temperature under the clothing to be controlled and/or adjusted. Therefore, the sensation of discomfort is reduced, but does not disappear.

In addition, under certain conditions of use, sufficient ventilation cannot be established, which makes this technique ineffective, and clearly not satisfying. This is particularly the case when the ambient temperature is very high, for example in certain industrial environments such as foundries, or in a fire. In fact it is well known that, under such conditions, the heat quickly becomes intolerable, when the skin temperature is not sufficiently regulated and is too high, and there is a risk of trauma and/or danger to the health, manifesting for example in the form of tissue damage and/or neurological sequelae.

Storing cold inside or on the surface of certain articles of clothing or accessories, such as vests or helmets, has also been contemplated to cool the wearer of the clothing or accessory.

Thus, for example, rigid molded helmets containing a fluid solution which freezes when placed in the freezer, are known.

One disadvantage of this technique is that contact with the head is uncomfortable when the helmet is put on, due to the fact that frost forms on the inner surface. In addition, it may sometimes lead to risks of scalp burn or complications associated with hypothermia.

Another disadvantage of this technique is that the helmet loses flexibility when it is frozen.

Still another disadvantage of this known technique is that the rigid shell that lines the inside of the helmet is not designed to absorb sweat, and that the skin does not “breathe” under the helmet, particularly due to the presence of a barrier formed by the layer of solidified solution.

Equipping a vest with pockets defining compartments in which removable pouches containing a cooled and at least partially solidified solution, for example a paraffin-based solution, has also been considered. When the paraffin has completely melted, the pouches can be replaced by new pouches.

This known technique presents several disadvantages.

Thus, one disadvantage of this known technique is that the frozen pouches stiffen the vest, which makes it cumbersome and uncomfortable.

On the other hand, the thickness of the pouches must be significant so as to guarantee a cooling effect for several hours, which hampers the movement of the arms.

Another disadvantage of this technique is the need to handle the pouches.

Another disadvantage of this known technique is also that it is complicated to implement, due to the care required when making the pockets.

### 4. DISCLOSURE OF THE INVENTION

An exemplary embodiment of the present disclosure relates to a device for covering and/or protecting at least one part of the outer surface of a human or animal body.

According to the invention, such a device comprises at least one fixed portion for cooling at least one region of said part of the outer surface of the body, capable of being cooled and/or frozen, and presenting at least three layers arranged in the following order:
a breathable layer intended to be applied on and/or near said region;
a flexible cold storage and restitution layer;
a heat insulation layer, intended to delay the heating of said cold storage and restitution layer.

Thus, the invention proposes, in an original way, integrating into or fixing to a device, such as an article of clothing or a clothing accessory, at least one cooling and flexible portion constituted of the cooperative assembly of at least three dedicated stacked layers, bringing local coolness to the wearer of the device, without sacrificing ergonomics and comfort and, if necessary, making a stay in certain very hot environments if not pleasant at least bearable.

Upon use, the device only has to be placed in the freezer for at least a few minutes and generally one to two hours, to cool the cold storage layer to a predetermined temperature of use. The presence of the breathable layer prevents frost from forming on the surface during this period.

When the device according to the invention is extracted from the freezer, it may be easily put on or put in place, due to the flexibility of the cooling portion. The cooled cooling portion then procures a cooling effect through the breathable layer that can be prolonged for several hours, thanks to the protection of the heat insulation layer, in particular.

According to at least one particularly advantageous embodiment of the invention, said breathable and/or said heat insulation layer comprises means for securing to at least one fourth layer, so as to maintain said fourth layer at least partially stacked on the said layer or layers.

When the device is taken from the freezer, the fourth layer, at ambient temperature, deposited on the breathable layer and/or the heat insulation layer, prevents the cooled surface of the cooling portion from being in direct contact with the skin, which prevents an unpleasant sensation and, in certain cases, burning of the cutaneous tissues.

According to a particular aspect of the invention, said securing means comprise at least one self-gripping element of the hook and loop fastener type.

Thus, thanks to removable self-gripping securing means, such as, for example, Velcro (registered trademark) type hooks and felt strips, the fourth layer can be simply attached to or detached from the cooling portion, as needed.

Preferentially, said fourth layer is a breathable layer.

Thus, the water vapor issued from perspiration or sweat can be evacuated through the fourth layer. It may also be provided that the material of the fourth layer is suitable for absorbing transpiration. In addition, it prevents frost from forming in contact with the skin.

Advantageously, said heat insulation layer comprises at least one layer belonging to the group comprising at least:
an air retention layer, comprising for example wadding;
a reflective layer, preferentially metalized;
a fire-proof and/or impermeable layer.

To be more effective, the heat insulation layer can be formed of a stack of one or more stacked air retention and/or reflective and/or fire-proof layers, alternating if necessary.

More specifically, multiplying the number of parallel reflective layers enables the efficacy of the insulation layer to be increased with regard to heat exchanges by thermal radiation. On the other hand, by aluminizing the reflective layers, a significant thermal reflection coefficient is obtained.

The use of wadding, in which the interlacing of fibers naturally forms an insulating air space, in the constitution of the air retention layer (thermally insulating air space) also presents many other advantages: it reassumes its shape after having been compressed, enables two reflective layers to be interconnected, is water-washable and constitutes an inexpensive material.

Advantageously, said insulation layer comprises at least one element constituted of Teflon.

Thus, the transmission of moisture through the insulation layer is limited.

Preferentially, said breathable layer is quilted.

The device is thus made more comfortable.

In at least one advantageous embodiment of the invention, said cold storage and restitution layer comprises a mixture of a substance containing water and a plurality of hydrophilic absorbers distributed in said substance, said substance separating said absorbers remaining deformable when said mixture is cooled to −18° C.

The composition of this mixture may advantageously be of the type as that described in document U.S. Pat. No. 5,800,491, for example, which remains pliant at −18° C.

In at least one particular embodiment of the invention, said mixture is contained in a plurality of compartments with distinct two-by-two contours.

Thus, thanks to compartments with suitable shapes, it is possible to simply cover the entire inner surface of the device.

Such a device may form an article of clothing, a covering or a patch.

It may for example be a suit, clothing, such as a headband, a medical covering or a blanket for horses or domestic animals.

It may also be an article of clothing belonging to the group comprising at least:
chest belt;
helmet;
cap;
bulletproof vest;
mini shorts or boxer shorts;
elbow support;
ankle support;
knee protector;
shoulder protector.

### 5. LIST OF FIGURES

The invention will be better understood upon reading the following description, given only by way of non-limiting example in reference to the attached drawings, in which:

FIG. 1 is a schematic view of a first mode of embodiment of a device according to the invention, worn as a chest belt by a cook;

FIG. 2 represents a partial cross-sectional view A-A of the device presented in FIG. 1;

FIG. 3 illustrates a second mode of embodiment of the invention used as a helmet, in an extended position;

FIG. 4 represents another mode of embodiment of the invention, taking the form of a medical covering;

FIG. 5 illustrates a horse blanket according to the invention.

### 6. DETAILED DESCRIPTION OF AN EMBODIMENT OF THE INVENTION

### 6.1. Restatement of the Principle of the Invention

The principle of the invention therefore is based on the implementation of non-removable cooling portions on covering and/or protecting devices such as articles of clothing or coverings, comprising at least one flexible layer containing a cooling solution inserted between an inner breathable layer and an outer heat insulation layer.

### 6.2. First Example of Embodiment of the Invention

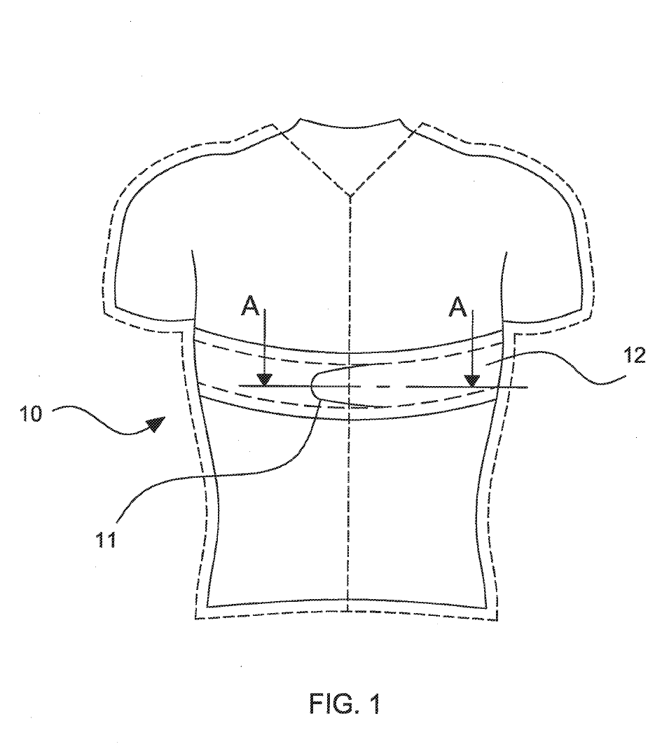

FIG. 1 is a partial view of the torso of a cook wearing under his jacket (represented for further clarity in dotted lines in FIG. 1) a first example of the covering and/or protecting device according to the invention, implemented in the form of a chest-lung belt 10, in order to keep cool when active, for example, close to an oven in the summertime.

This belt 10 provides a cooling effect for at least two hours and preferentially four hours, after being removed from a freezer in which it had been placed for a period sufficient for cooling to −18° C.

It is noted that due to its slim thickness, this belt 10 can be worn in the context of many other professional activities, for example when deep sea diving.

Belt 10 advantageously presents a universal section, or in other words is suitable for any morphology and can be used by left-handed users and right-handed users both.

The position of this belt 10 is also vertically adjustable. For this purpose, it is equipped with removable closing means of a known type, such as for example a Velcro (registered trademark) type felt and hook strip 11 in this first embodiment of the invention. Snaps, safety pins or any other similar means may also be provided in variations of this embodiment.

As can be seen in FIG. 1, a skin cooling portion 12 is sewn onto the entire length of belt 10.

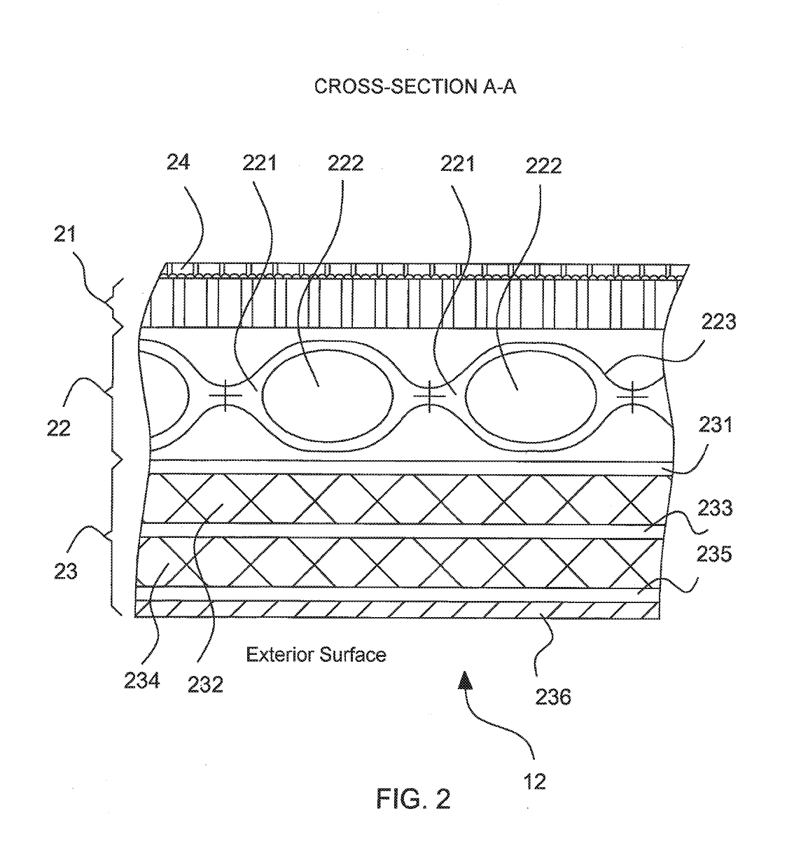

In the partial cross-sectional view A-A of belt 10, represented in FIG. 2, it may be observed that portion 12 is constituted of three distinct successive layers 21, 22 and 23, which are stacked.

When belt 10 has been removed from the freezer, the inner face of portion 12 was then covered, in this embodiment, by a fourth layer 24, kept at ambient temperature. This layer 24 eliminates, or at least greatly reduces, the unpleasant and possibly painful effect of contact of a cold and/or locally frosted surface with the skin, when the belt is applied to the chest. In addition, it presents holes or mesh distributed over its surface, so as to form a “breathable” surface.

Layer 24 is removably fixed to layer 21 by self-gripping means, For example hook fasteners provided on one of its faces secured to corresponding loop fasteners provided for this effect on the surface of layer 21. Therefore it may easily be removed from portion 12, when the cooling effect has stopped, before the belt is placed back in the freezer.

In FIG. 2, the inner face of portion 12 is formed of a first breathable layer 21 of open-weave mesh presenting passages to enable perspiration and transpiration to be evacuated.

This first layer 21 is for example obtained in a machine-washable pique knit, composed of 60% polyester and 40% cotton and surface-treated to prevent sweat stains. In addition, it is quilted for comfort and preferably meets class F1 (standard NF) fume toxicity criteria.

The thermal resistance of layer 21 was provided with a value sufficient for preventing the proximity of the second cold storage and restitution layer 22 with the skin from creating an unpleasant sensation through this layer 21.

The second layer 22 is formed by aligning cooling pliant pouches 221, for example by the FirstIce (registered trademark) brand, slid inside compartments 222 of a sleeve 223 in a textile material. These compartments 222, substantially attached two-by-two, are sealed by stitching so as to permanently hold the pouches 221.

Each of pouches 221 contains a gelatinous mixture composed of a substantially liquid solution constituted of 85 weight percent water and 15% humectants, such as propylene glycol, ethylene glycol, glycerin, dimethyl sulfoxide, dimethyl formamide or a humectant from their combination, and a plurality of hydrophilic absorbers distributed in the solution.

This humectant reduces the mobility of water outside of hydrophilic absorbers, each water volume then freezes, forming a dispersed flake. A global consequence is thus that below 0° C., and at least to −18° C., the humectant prevents the water from forming a compact rigid mass connecting the absorbers.

The third layer 23 insulates the body from external hot air and prevents a too-significant loss of cold by the cold storage layer 22. This layer is advantageously made from a single piece to prevent the “thermal bridge” phenomenon.

In this embodiment, layer 23 is constituted of alternating aluminized thermal radiation reflective layers 231, 233 or 235 with air retention layers 232 or 234 formed of wadding in order to adequately fight heat transports by radiation and convection. Rough bumps, obtained for example by stamping, are provided on the face of the aluminized layers facing layer 22, so as to facilitate the fixation of air retention layers.

Layer 23 is covered by an outer layer of fireproof fabric 236 treated with Teflon, to limit moisture retention.

In a variation of this embodiment, making layer 236 water- and steam-impermeable may be contemplated, to prevent ambient moisture from penetrating to the layers of wadding.

### 6.. Second Example of Embodiment of the Invention

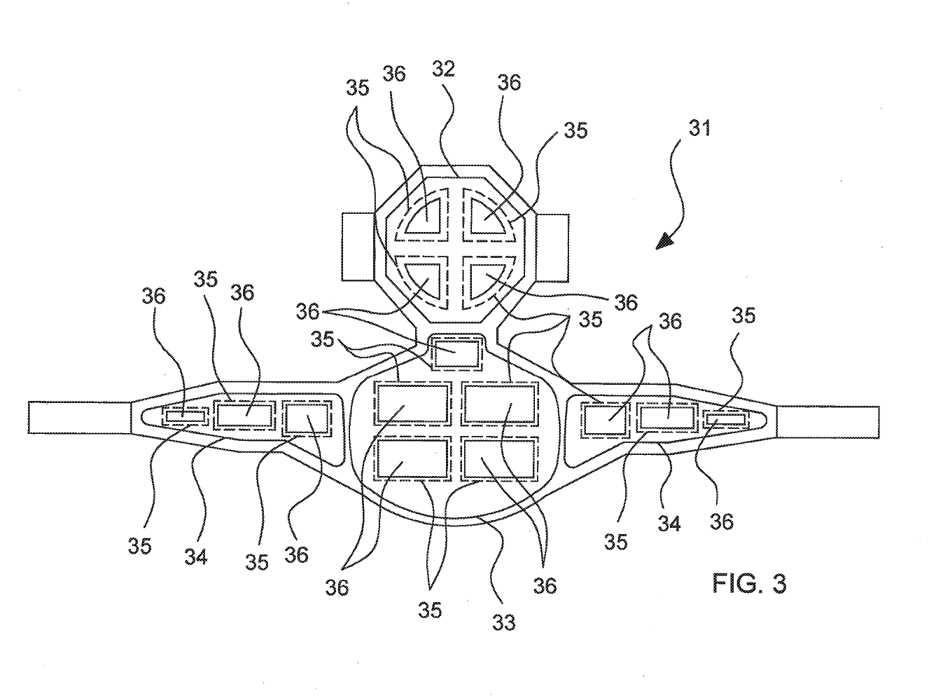

A second example of embodiment of the invention is presented in reference to FIG. 3.

The device according to the invention is in this example a flexible protective helmet 31, represented extended flat on its outer face, in FIG. 3.

This helmet 31 presents fixed cooling portions 32, 33, 34, that are distinct two-by-two. To maximize the cooling surface in contact with the head, these portions 32, 33, 34 are distributed over the entire inner surface of the helmet, and present complex cutouts so as to substantially follow the contours of the helmet.

Each of these portions is formed in its thickness of three stacked layers:
a layer in a breathable material, such as for example a polyester fabric of the Coolmax (registered trademark) brand;
a flexible cold storage and restitution layer inserted between the breathable layer and a heat insulation layer;
a heat insulation layer, presenting an inner structure substantially identical to that of the belt described previously.

It should be noted that the shape and number of compartments and the cooling fluid solution pouches 36, that they house, have advantageously been modified to adapt to the complex contours of portions 32, 33 and 34. These flexible pouches 36 are preferentially from the FirstIce (registered trademark) brand.

### 6.4. Third Example of Embodiment of the Invention

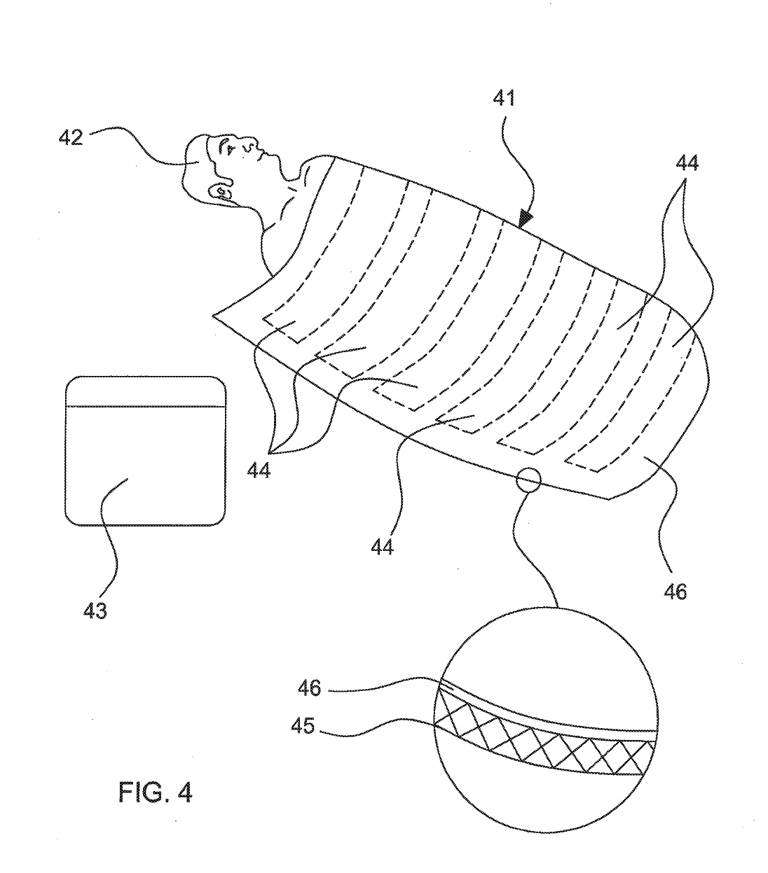

Another embodiment of the invention is schematically illustrated in FIG. 4. In this FIG. 4, a medical covering 41 according to the invention is represented, placed by first aid practitioners on the body of a person 42 who is a victim of sunstroke or hyperthermia, to cool the body rapidly.

It had been removed from a cooler 43 transported by the emergency medical response team, in which it was kept at a negative temperature, for example −12° C., and spread directly onto the body 42 of the patient.

Covering 41 is constituted of a plurality of cooling bands or portions 44 of the type as those described in detail above in relation to the first example of embodiment of the invention, sewn onto a textile piece 45.

The upper face of this covering is also formed of an aluminized Mylar film 46 to protect the victim 42 from ambient heat.

It should be noted that due to the flexibility of the cooling portion, the covering has a flexibility that allows it to be stored folded in the cooler, and to be used after it is removed from the cooler. In addition, this flexibility allows the covering to be easily handled, to allow it to follow the shape of the body of the patient.

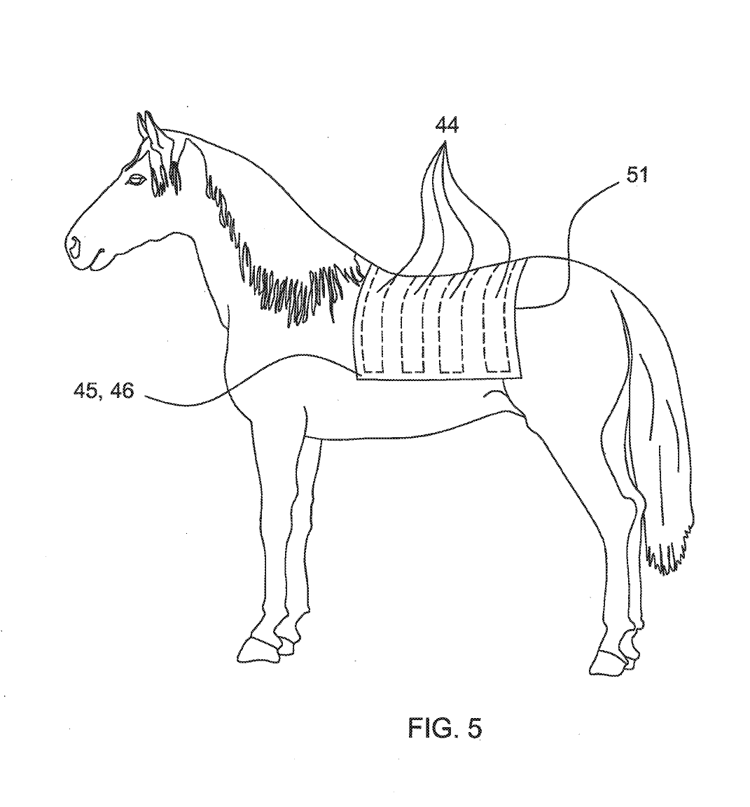

In a variation of this embodiment, making the invention available in the form of a horse blanket 51 may be contemplated (see FIG. 5), for example to cool a horse down after a horse race.

### 6.5. Optional Characteristics and Other Advantages of the Invention

In the variations of embodiments described in detail above, modifying the composition and number of layers can of course also be contemplated. For example, the fourth layer can be folded to allow it to be secured to both the breathable layer and the heat insulation layer.

A device according to the invention may also be implemented, for example, in the form of a:
cap;
mini shorts or boxer shorts;
bulletproof vest;
elbow support;
ankle support;
knee protector;
shoulder protector;
sheet;
patch;
covering for a domestic animal.

An exemplary embodiment of the present disclosure overcomes at least some of the disadvantages of the techniques from the prior art.

More specifically, an embodiment provides a device, such as for example, an article of clothing or a covering, that is effective.

In particular, at least one particular embodiment provides a technique that is appropriate in the event of exposure to very high temperatures and/or to fire.

An embodiment obtains such a device that is comfortable, compact (with a slim thickness, in particular) and light.

An embodiment also proposes a technique that is simple to implement and to use.

Although the present disclosure has been described with reference to one or more examples, workers skilled in the art will recognize that changes may be made in form and detail without departing from the scope of the disclosure and/or the appended claims.

1. A device for covering and/or protecting at least one part of the outer surface of a human or animal body, wherein the device comprises:
at least one fixed portion for cooling at least one region of said part of the outer surface of the body, able to be refrigerated and/or frozen, and presenting at least three layers disposed in the following order:
a breathable layer intended to be applied on and/or near said region;
a flexible cold storage and restitution layer;
a heat insulation layer, configured to delay the heating of said cold storage and restitution layer.

2. The device according to claim 1, wherein at least one of said breathable layer or said heat insulation layer comprises means for securing with at least one fourth layer, so as to maintain said fourth layer stacked at least partially on at least one of the at least three layers.

3. The device according to claim 2, wherein said means for securing comprises at least one self-gripping element of a hook and loop fastener type.

4. The device according to claim 2, wherein said fourth layer is a breathable layer.

5. The device according to claim 1, wherein said heat insulation layer comprises at least one layer belonging to the group consisting of:
an air retention layer, comprising for example wadding;
a heat reflective layer, preferentially aluminized;
a fire-proof and/or impermeable layer.

6. The device according to claim 1, wherein said insulation layer comprises at least one element constituted of Teflon.

7. The device according to claim 1, wherein said breathable layer is quilted.

8. The covering and/or protecting device according to any one of claim 1, wherein said cold storage and restitution layer comprises a mixture of a substance containing water, and a plurality of hydrophilic absorbers distributed in said substance, said substance separating said absorbers remaining deformable when said mixture is cooled to −18° C.

9. The device according to claim 8, wherein said mixture is contained in a plurality of compartments with distinct two-by-two contours.

10. The device according to claim 1, wherein the device forms an article of clothing, a covering or a patch.

11. The device according to claim 10, wherein said article of clothing belongs to the group consisting of:
chest belt;
helmet;
cap;
mini shorts or boxer shorts;
bulletproof vest;
elbow support;
ankle support;
knee protector;
shoulder protector.
